(19) United States
(12) Patent Application Publication
(10) Pub. No.: US 2013/0164233 A1
(43) Pub. Date: Jun. 27, 2013

(54) SKIN-WHITENING ESSENCE COMPOSITION CONTAINING TYROSINASE INHIBITOR

(71) Applicant: Ming-Chen LEE, Taipei City (TW)

(72) Inventor: Ming-Chen LEE, Taipei City (TW)

(21) Appl. No.: 13/336,180

(22) Filed: Dec. 23, 2011

(51) Int. Cl. A61K 8/49; A61Q 19/02

(52) U.S. Cl. 424/62

(57) ABSTRACT

A skin-whitening essence composition containing a tyrosinase inhibitor is disclosed. The skin-whitening essence composition includes: 0.1-2.5 wt % of the tyrosinase inhibitor (CitrusC), 0.1-2.5 wt % of ascorbic acid 2-glucoside (AA2G), 0.1-2.5 wt % of tranexamic acid and a moisturizing essence base that work synergistically to provide the skin-whitening essence composition with skin-whitening effect.

### BACKGROUND OF THE INVENTION

1. Technical Field

The present invention relates to compositions for skincare, and more particularly, to a skin-whitening essence composition containing a tyrosinase inhibitor, being able to inhibit spontaneous polymerization of tyrosinase, thereby preventing synthesis of melanin in skin.

2. Description of Related Art

Not limited to sunny summer days, it's almost an everyday work for Asian women to fight sunshine and pursue skin whitening. As revealed in an A.C. Nielsen survey, Taiwan is second only to Korea all over the world as a place where women are fond of white skin.

Referring to FIG. 1, synthesis of melanin in melanosome relies on many enzymes for catalysis, in which the most important is tyrosinase. Tyrosinase first hydroxylates tyrosine into L-3,4-dihydroxyphenylalanine (L-dopa) and DOPA then is rapidly converted into dopaquinone, which is an intermediate exists as watershed along the course of melanin's formation. With the presence of cysteine or glutathione that provides sulfhydryl groups, dopaquinone can be converted into cysteinyldopa, and then oxidized and polymerized into soluble, yellow-brown pheomelanin. On the other hand, without the provision of sulfhydryl groups, dopaquinone can spontaneously get cyclized and become dopachrome as an orange intermediate. Dopachrome spontaneously losing its carboxyl groups generates 5,6-dihydroxyindole (DHI), which can be rapidly oxidized and polymerized into insoluble, high-molecular-weight, dark-brown DHI-melanin. Differently, when there is dopachrome tautomerase, dopachrome does not lose its carboxyl groups, and is converted into DHI-2-carboxylic acid (DHICA), which is then oxidized and polymerized into light-brown, slightly soluble, middle-sized DHICA-melanin. DHI-melanin and DHICA-melanin are both members of eumelanins, and melanin includes eumelanins and pheomelanins.

As new whitening agents have been continually found and applied, the Oriental cosmetics prefer the materials extracted from various plants while the Western cosmetics often made from what is obtained biotechnologically. Nevertheless, there are some whitening agents welcome by both of the markets, such as levorotatory Vitamin C, Vitamin C derivatives, kojic acid, arbutin, Vitamin A, alpha hydroxy acids and some plant extracts. Those pharmaceutical-grade whitening agents, like hydroquinone, azelaic acid and retinoic acid, shall be used under doctors' instructions and prescriptions. The aforementioned whitening agents are mostly competitive inhibitors for tyrosinase, and function by anticipating tyrosinase in acting with tyrosine, such that tyrosinase has no chance to act with tyrosine, and the formation of melanin can be eliminated.

However, these competitive inhibitors for tyrosinase can only function under low-pH conditions, and cannot inhibit melanin's formation unless they are provided in a concentration high enough to anticipate tyrosinase completely. It is problematic because higher concentration means greater irritation, which is responsible for skin allergy and disorder. In addition, these competitive inhibitors for tyrosinase are mostly sensitive to light. As they are unstable when receiving light, browning (e.g. arbutin and kojic acid) or yellowing (e.g. ascorbic acid and derivatives thereof) tends to happen and causes light-exposed skin-whitening products to yellow and black. This character is unfavorable to shelf stability and adds difficulty to transportation and storage of products made therefrom.

There are also skin-whitening products each coming with two parts, namely one part for day use and the other part for night use, which contain different ingredients. The part for day use is mainly composed of ingredients for sun block, in order to reduce direct exposure of human skin to UV rays in daytime, thereby reducing the formation of melanin, while the part for night use is aimed at repairing and whitening skin. Such dualistic products nevertheless cause inconvenience to manufacture and distribution. Moreover, misuse of the functionally reverse parts may weaken the effect of the products and, even worse, bring about adverse effects.

### SUMMARY OF THE INVENTION

For remedying the problems as addressed above, the present invention provides a skin-whitening essence composition containing a tyrosinase inhibitor. The essence composition is advantaged by having its ingredients easy to obtain and inexpensive, having optimum reaction under environment of from pH7 to pH9, being unlikely to cause skin allergy and disorder, being stable to light, and being easy to transport and store.

According to the present invention, a skin-whitening essence composition containing a tyrosinase inhibitor comprises:

0.1-2.5 wt % of the tyrosinase inhibitor (CitrusC);

0.1-2.5 wt % of ascorbic acid 2-glucoside;

0.1-2.5 wt % of tranexamic acid; and

a moisturizing essence base that work synergistically on whitening skin.

It is expectable that the tyrosinase inhibitor (CitrusC), ascorbic acid 2-glucoside and tranexamic acid in the inventive composition can work synergistically on whitening skin with the best whitening effect achieved by the end of the first week of its use, and with sustained effect of reducing melanin concentration over long-term (consecutive two weeks or longer) use.

Another expectable feature of the present invention is that the tyrosinase inhibitor (CitrusC) is employed as an effective whitening component of the disclosed composition, wherein for the purpose of the present invention the tyrosinase inhibitor (CitrusC) is preferably with a pH value of 7-9, for the optimal reaction.

Still another expectable feature of the present invention is that the tyrosinase inhibitor (CitrusC) used in the disclosed composition is a non-competitive inhibitor, and thus can be effective in inhibiting the formation of melanin even with a relatively low concentration.

Yet another expectable feature of the present invention is that the tyrosinase inhibitor used in the disclosed composition possesses high light stability, so the composition is unlikely to discolor due to its exposure in light, eliminating the need of protecting the composition with special light-shielding packaging.

### BRIEF DESCRIPTION OF THE DRAWINGS

The invention as well as a preferred mode of use, further objectives and advantages thereof will be best understood by reference to the following detailed description of an illustrative embodiment when read in conjunction with the accompanying drawings, wherein:

FIG. 1 illustrates the formation of melanin;

FIG. 2 is a flowchart of an experiment conducted for demonstrating the effect of the disclosed composition;

FIG. 3 is a first graph exhibiting the positive effect (%) of different groups of samples;

FIG. 4 is a second graph exhibiting the positive effect (%) of different groups of samples; and

FIG. 5 a graph exhibiting the whitening intensity (%) of different groups of samples.

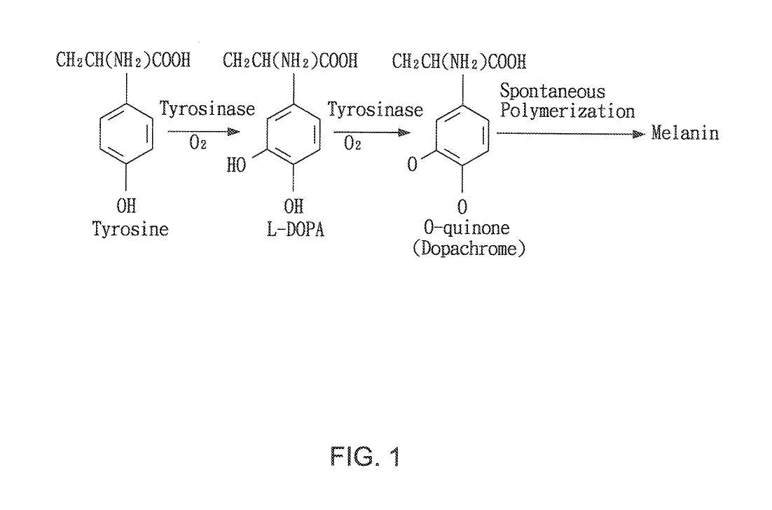

### DETAILED DESCRIPTION OF THE INVENTION

The inventor of the present invention has been previously granted with U.S. Pat. No. 7,125,572 (the same invention has also been patented in Taiwan as Taiwan Patent No. 1313177). The foregoing patent involves preparing a tyrosinase inhibitor extract from lemon peels by mixing 100 g of freshly minced lemon peels with 100-1000 ml of propylene glycol, breaking cell walls of the lemon peels with 1-59 seconds of sonication, sterilizing the resultant solution with microwave and removing the lemon peels with centrifugation, so as to obtain the isolated supernate as the patented substance. The yield of 100 g of lemon peels extracted with propylene glycol is 70% to 90% of the volume of propylene glycol used.

The patented substance, as a tyrosinase inhibitor extracted from lemon peels, has been named by the inventor as CitrusC, which contains protein, peptide, isoflavone and flavonoid. While it is believed that the protein is the main active component for inhibiting tyrosinase, the other components are likely to provide either additive effect or anti-aging effect, so the tyrosinase inhibitor extract does not need extreme purification, therefore saving manufacturing costs.

The tyrosinase inhibitor (CitrusC) as disclosed in U.S. Pat. No. 7,125,572 features the following characteristics:

1. Being a non-competitive inhibitor, which can effectively inhibit the formation of melanin with a relatively low concentration;

2. Presenting high inhibiting capability against tyrosinase at 25-37° C., and particularly exhibiting its maximum activity at about 25° C., while remaining 80% inhibiting capability against tyrosinase even at about 37° C. (human body temperature);

3. Reacting optimally at pH 7-9, where the tyrosinase inhibitor extract is safe and brings no irritation or allergy to the skin when applying to skin;

4. Possessing a high level of light stability, which ensures the inhibitor not to discolor under light exposure, and facilitates the inhibitor's transportation and storage by eliminating the need of special light-shielding packaging;

5. Being capable of reducing O-quinone, which is an intermediate generated during the synthetic process of melanin, thereby obstructing the synthesis of melanin and enhancing whitening effect; and

6. Being made from citrus peels, meaning that the raw material is easy to obtain.

The patented inhibitor is extracted from citrus peels and made through biochemical reaction. The patented inhibitor contains mainly flavonoid and the peptide, wherein the peptide is the main active component for inhibiting tyrosinase and augmenting the other whitening ingredient while flavonoid provides anti-aging and anti-irritation effects. The tyrosinase inhibitor extract does not need extreme purification, therefore saving manufacturing costs.

The present invention thus uses the tyrosinase inhibitor disclosed in U.S. Pat. No. 7,125,572 (Taiwan Patent No. 1313177) as the active ingredient to develop a series of skin-whitening products and the human subject research has been conducted to prove the whitening effect of these products.

### Materials and Methodology

### I. MATERIALS

(1) 300 samples for testing whitening efficiency were prepared and grouped into the following groups:

1. Group A: Moisturizing Essence Base (BK moist essence) +Tranexamic Acid (50 samples)

2. Group B: Moisturizing Essence Base+Tyrosinase Inhibitor (CitrusC) (50 samples)

3. Group C: Moisturizing Essence Base+Ascorbic Acid 2-Glucoside (AA2G) (60 samples)

4. Group D: Moisturizing Essence Base+Tranexamic Acid+Ascorbic Acid 2-Glucoside (AA2G)+Tyrosinase Inhibitor (CitrusC) (80 samples)

5. Group E: Vital Purity Lotion (Nature & Co, Kose) (30 samples), with a market price about USD. 0.1/ml

6. Group F: CyberWhite Brilliant Cells Full Spectrum Brightening Essence (Estee Laude) (30 samples), with a market price about USD. 3.5/ml

(2) Ingredients and Formulas:

The ingredients of samples for Groups A, B, C and D are listed in Table 1. The moisturizing essence base contains at least one moisturizing component selected from aloe vera extract, olive oil PEG-7 esters, γ-polyglutamic acid, lavender flower extract and combination thereof. The ingredients of samples for Groups E and F are listed in Tables 2 and 3, respectively.

(3) For preventing the subjects and testers from being affected by individual psychological expectation, the 300 samples were randomly allotted with serial numbers from No. 1 to No. 300.

TABLE 1
Ingredients of Samples for Groups A, B, C and D

| | | Group | | | |
|---|---|---|---|---|---|
| Ingredient | Function | A (%) | B (%) | C (%) | D (%) |
| Aloe Vera | Moisturizing, | 10 | 10 | 10 | 10 |
| Extract | replenishing | | | | |
| Olive Oil | Moisturizing | 2 | 2 | 2 | 2 |
| PEG-7 Esters | | | | | |
| γ-Polyglutamic | Moisturizing | 1 | 1 | 1 | 1 |
| Acid | | | | | |
| Lavandula | Moisturizing, | 1 | 1 | 1 | 1 |
| Angustifolia | replenishing | | | | |
| (Lavender | | | | | |
| Flower | | | | | |
| Extract) | | | | | |
| Yeast Extract | Yeast extract | 1 | 1 | 1 | 1 |
| Urea (Merck) | Urea | 1 | 1 | 1 | 1 |
| Honey (LIPO | Honey extract, | 1 | 1 | 1 | 1 |
| HON) | water-soluble | | | | |
| Citrus unshiu | Low-molecular- | — | 0.1-2.5 | — | 0.1-2.5 |
| Peel Extract | weight peptide, | | | | |
| | Tyrosinase inhibitor | | | | |
| Ascorbic Acid | Anti-oxidant | — | — | 0.1-2.5 | 0.1-2.5 |
| 2-Glucoside | | | | | |
| Tranexamic | Anti-allergy, | 0.1-2.5 | — | — | 0.1-2.5 |
| Acid | anti-irritation | | | | |
| Dimethylol | Liquid preservative | 0.35 | 0.35 | 0.35 | 0.35 |
| Dimethyl | | | | | |
| Xanthan Gum | Thickener, | 0.2 | 0.2 | 0.2 | 0.2 |
| | plant-derived | | | | |
| | colloidal | | | | |
| | polysaccharide | | | | |
| Allantoin | Skin cell proliferant | 0.2 | 0.2 | 0.2 | 0.2 |
| EDTA | Metal chelating agent | 0.03 | 0.03 | 0.03 | 0.03 |
| Methylparaben | Preservative | 0.1 | 0.1 | 0.1 | 0.1 |

| | |
|---|---|
| Aqua | Sufficient to make the final |
| | composition up to 100 |
| | percent |

TABLE 2
Ingredients of Vital Purity Lotion
from Nature & Co, Kose (Group E)

| Ingredient | Feature |
|---|---|
| Water/Aqua | Solvent |
| Butylene Glycol | Solvent, moisturizer |
| Alcohol | Solvent |
| Glycerin | Solvent, moisturizer |
| Aminobutyric Acid | Hair |
| | conditioning agent |
| Chamomilla Recutita Flower | Fragrance, Calming, |
| Extract | allaying |
| Lavandula angustifolia Flower | Calming, allaying |
| Extract | |
| Sodium PCA | Antistatic agent, |
| | moisturizer |
| Succinoyl Atelocollagen | Emollient |
| Tocopherol | Anti-oxidant |
| Citric Acid | Peeling, toner |
| Disodium EDTA-2Na | Metal chelating agent |
| PEG-50 Hydrogenated Castor | Emulsifier, |
| Oil Isostearate | lipophilic thickener, |
| | fragrance |
| | dispersing agent |
| Sodium Citrate | pH controlling agent, |
| | anti-oxidant |
| Sodium Lactate | Moisturizer, |
| | balancing pH |
| Ethylparaben | Preservative |
| Methylparaben | Preservative |
| Fragrance (PARFUM) | Fragrance |

TABLE 3
Ingredients of Estee Laude CyberWhite Brilliant Cells
Full Spectrum Brightening Essence (Group F)

| Ingredient | Feature |
|---|---|
| Water/Aqua | Solvent |
| EAU, Dimethicone | Providing smoothness in use |
| Polysorbate 40 | Hydrophilic emulsifier, |
| | surfactant, fragrance dispersing |
| | agent |
| Pentylene glycol | Small-molecule humectant, |
| | Emollient, bacteriostat |
| Acorbyl glucoside | Whitening agent, Anti-oxidant |
| Yeast extract/Faex/Extrait de | Containing enzymes, various |
| levure | vitamins, minerals and |
| | saccharides, being helpful to |
| | nurse and nourish skin |
| Polyacrylamide | High-molecular-weight polymer, |
| | thickener, Antistatic agent, highly |
| | absorbent |
| Myristyl alcohol | Skin emollient, emulsification |
| | stabilizer, thickener, viscosity |
| | controlling agent |
| Acetyl glucosamine | Moisturizing, replenishing |
| Sucrose | Small-molecule moisturizer |
| PEG-10 Dimethicone | Skin emollient |
| Phenoxyethanol | Preservative, perfume fixative |
| C 13-14 Isoparaffin | Thickener |
| Titanium dioxide (CI77891) | Titanium dioxide dispersing agent |
| Sorbitol | Small-molecule humectant, |
| | thickener |
| Sodium hydroxide | Adjusting ph value |
| Butylene glycol | Solvent, moisturizing |
| MICA | Emollient, coloring agent |
| Propylene glycol dicaprate | Skin emollient |
| Caffeine | Anti-irritation, promoting |
| | metabolism and blood circulation |
| Tocopheryl acetate | Moisturizing, nourishing, |
| | protecting skin from UV |
| Algae extract | Promoting metabolism, |
| | moisturizing, softening, reducing |
| | inflammation and calming skin, |
| | anti free radical |
| Laureth-7 | Emulsifier, surfactant |
| Pantethine | Vitamin B5 derivative, softener, |
| | moisturizing, softening |
| Chlorphenesin | Preservative, bactericide |
| Glycyrrhetinic acid (K2) | Anti-irritation, skin conditioning |
| | agent |
| Bifidus ferment filtrate | Moisturizing, replenish |
| Sodium RNA | Skin conditioning agent |
| O-Cymen-5-ol | Bacteriostat |
| Glycine soja (soybean) seed | Softener, humectant |
| extract | |
| Fragrance (Parfum) | Fragrance |
| Gentianalutea (Gentian) root | Calming, allaying |
| extract | |
| Phytosphingosine | Moisturizing and improving skin |
| | immunization |
| Sodium hyaluronate | Strong humectant |
| Helianthus annuus (Sunflower) | Strengthening skin defense |
| seedcake | |
| Silica | Viscosity reguator |
| Citric acid | Peeling, toner |
| Whey protein/lactis protein/ | Tightening and smoothening skin |
| proteine du petit-lait | |
| Chamomilla recutita | Calming, allaying, antiseptic, |
| (matricaria) flower extract | astringent |
| Disodium EDTA-2Na | Metal chelating agent |
| Oryza sativa (Rice) bran | Moisturizing, anti-irritation, |
| extract | enhanced whitening effect |
| Hordeum vulgare (Barley) | Skin emollient |
| extract/extrait d'orge | |
| Laminaria saccharina extract | Skin conditioning agent |
| Hydrolyzed rice bran extract | Calming and whitening skin |
| Plankton extract | Skin conditioning agent |
| Lecithine | Natural surfactant, moisturizer, |
| | anti-oxidant |
| Cucumis sativus (Cucumber) | Moisturizing, reducing |
| fruitextract | inflammation and calming skin, |
| | and whitening |
| Pyrus malus (Apple) fruit | Moisturizing, reducing |
| extract | free-radical incurred harm and |
| | anti-aging |
| Pueraria lobata root extract | Moisturizing, anti-irritation |
| Scutellaria baicalensis root | Reducing inflammation and |
| exract | calming skin, whitening |

### II. DESIGN OF EXPERIMENT

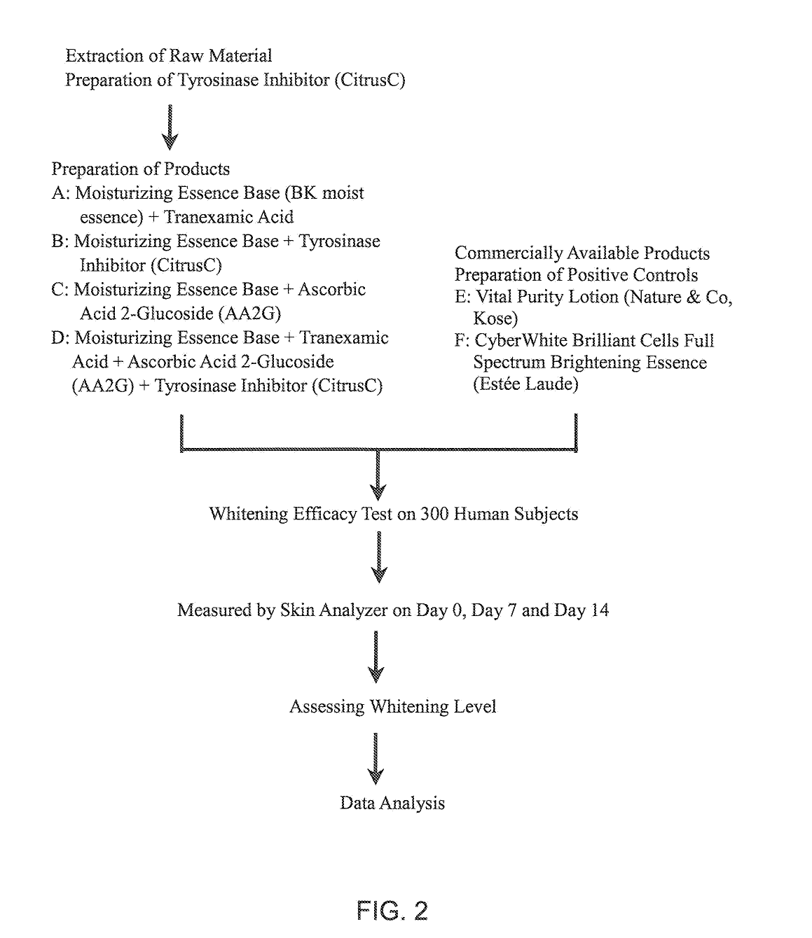

(1) The experiment was based on and modified from an experiment proposed by Sederma, the subsidiary of Merck KGaA. The flowchart of the designed experiment is shown in FIG. 2.

(2) Apparatuses

Computer: ASUS UL30A

System: Joy Silky

Device: Joy Silky Skin Analyzer provided by SILKY

### INDUSTRIES LIMITED

(3) Subjects for Experiment

300 individuals (including both males and females) having ages between 18 and 30 and having healthy skin were selected from students and teachers of National Pingtung University of Science and Technology (Pingtung, Taiwan) as the subjects.

(4) Methodology

Each of the subjects had the initial skin conditions at his/her backs of both hands measured by the Skin Analyzer (Day0). The subject's left and right hands were defined as a treated area and a control area, respectively. The back of the left hand was treated with a grain-size amount of the designated sample twice a day (day and night), while the back of the right hand was not treated. At the end of the first week (the 7^{th} day), the subjects were measured for recording the results of 7-day use (D7), and at the end of the second week (the 14^{th} day), the subjects were measured for recording the results of 14-day use (D14).

(5) Expression of Data

The figures were recorded as numerals from 1 to 100. The smallest numeral represents the lowest melanin concentration, and the greatest numeral represents the highest melanin concentration.

(6) Quantitative Assessment Formulas for Whitening Level

1. The whitening level achieved in the seven days of the first week from the commencement of using the sample (T0−7):

T0−7=[(D0−D7)−(D′−D′7)]/[(D0+D′0)/2]×100%

2. The whitening level achieved in the seven days of the second week from the commencement of using the sample (T7−14):

T7−14=[(D7−D14)−(D′7−D′14)]/[(D7+D′7)/2]×100%

3. The whitening level achieved in the total fourteen days of the consecutive two weeks from the commencement of using the sample (T0−14):

T0−14=[(D0−D14)−(D′0−D′14)]/[(D0+D′0)/2]×100%

D0: The initial skin condition of the treated area (back of left hand) on Day 0

D7: The skin condition of the treated area (back of left hand) on Day 7

D14: The skin condition of the treated area (back of left hand) on Day 14

D′0: The initial skin condition of the control area (back of right hand) on Day 0

D′7: The skin condition of the control area (back of right hand) on Day 7

D′14: The skin condition of the control area (back of right hand) on Day 14

4. Where the results of the three formulas above are regarded positive, it means that the sample was effective in whitening the subject's treated area. By summing up the number of samples presenting positive values in each sample group and dividing it by the effective value, the positive effect (%) was obtained.

### III. RESULTS AND ANALYSIS

This experiment was aimed at investigation into the whitening effect of the samples having different ingredients on human skin.

The samples to be tested were grouped into six groups. The samples of Group A were made from a moisturizing essence base added therein with 0.1-2.5% of a tranexamic-acid-based anti-allergy component. The samples of Group B and Group C were made from the moisturizing essence base added with 0.1-2.5% of the tyrosinase inhibitor (CitrusC) whitening component and 0.1-2.5% of ascorbic acid 2-glucoside (AA2G) as an anti-oxidant, respectively. The samples of Group D contained the moisturizing essence base and three more components, namely 0.1-2.5% of tranexamic acid, 0.1-2.5% of the tyrosinase inhibitor (CitrusC) and 0.1-2.5% of ascorbic acid 2-glucoside (AA2G). The samples of Group E and Group F were commercially available Vital Purity Lotion (sold in an open-shelf fashion) and CyberWhite Brilliant Cells Full Spectrum Brightening Essence (sold through cosmetics counters in department stores). Group E and Group F were provided as positive controls in the experiment for the tested samples to compare. The ingredients of the samples of Group E and Group F are listed in Table 2 and Table 3, respectively. The formula of Group E did not contain any effective whitening agent, as its claim was mainly focused on moisturization. The formula of Group F contained ascorbic acid 2-glucoside (AA2G) as a whitening component together with other skin-whitening plant extracts, such as oryza sativa (Rice) bran extract, hydrolyzed rice bran extract, cucumis sativus (Cucumber) fruitextract and scutellaria baicalensis root exract.

The 300 subjects of the experiment were selected from students and teachers of National Pingtung University of Science and Technology (Pingtung, Taiwan). The ratio of male subjects to female subjects was 1:3, with all the subjects having their ages between 18 and 30. The essential condition for the subjects to be selected was healthy skin.

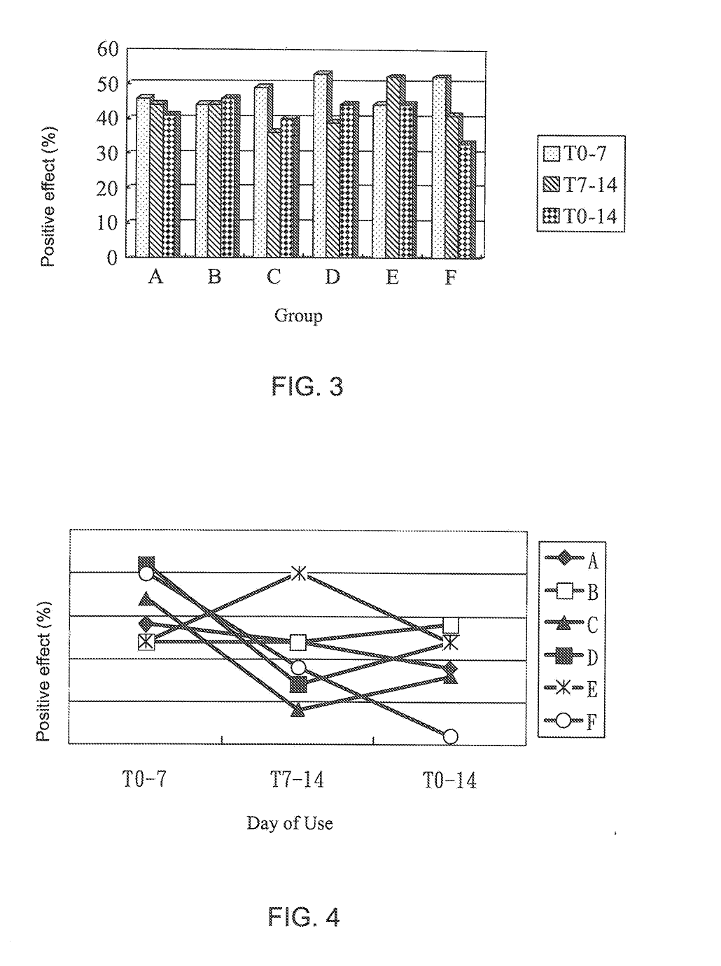

Table 4 includes data reflecting whitening efficiency of moisturizing essences with different formulas. As can be seen in FIG. 3, for the samples of Group A containing moisturizing essence base and 0.1-2.5% of tranexamic acid, in the 20 subjects, 46% of them experienced whitening effect after 7 days of use, and 20% more of them experienced whitening effect in the second week, while the continuous use of 14 days only gave whitening effect to about 35% of these subjects. Hence, although the samples of Group A whitened many subjects in the first week, it seemed to be the result of the skin's instinctive reaction to external substance. As the use continued, the ratio of the subjects who saw whitening effect became even lower. The results shown in this group seem to be psuedo whitening effect.

In the 24 subjects using the samples of Group B, 42% of them experienced whitening effect after one-week use, and the ratio increased by 4% in the period from Day 7 to Day 14, while the continuous use for the two whole weeks gave the whitening effect to 46% of these subjects. This indicates that the whitening essences containing 0.1-2.5% of tyrosinase inhibitor (CitrusC) made nearly 45% of the subjects have reduced melanin concentration. In other words, the tyrosinase inhibitor (CitrusC) was proven to be effective in providing stabile whitening effect even although it failed to continuously increase the whitening effect.

The samples of Group C were composed of the moisturizing essence base added with 0.1-2.5% of ascorbic acid 2-glucoside (AA2G). After the first week of use, the samples of this group exhibited the whitening effect on 50% of the subjects. After the second week of use, 8% more of the subjects also saw the whitening effect. At the end of two-week continuous use, 54% of the subjects remained seeing the whitening effect. The results indicate that the essence containing ascorbic acid 2-glucoside (AA2G) only provided meaningful whitening effect in the second week, and prolonged use did not show increased effectiveness. It is thus believed that the moisturizing essence base incorporating only ascorbic acid 2-glucoside (AA2G), when used over an extended term, would have the lasting of its whitening effect substantially limited because ascorbic acid 2-glucoside (AA2G) acted mainly as an anti-oxidant.

The whitening essence samples of Group D contained the tyrosinase inhibitor (CitrusC), ascorbic acid 2-glucoside (AA2G) and tranexamic acid, each in the amount of 0.1-2.5%. After seven days of using the samples of Group D, the whitening effect was exhibited on 62% of the subjects, being the highest ratio among the four groups (A, B, C and D) of samples. In other words, the samples containing the tyrosinase inhibitor (CitrusC), tranexamic acid, and ascorbic acid 2-glucoside (AA2G) did provide multiplied whitening effect, with the broader extent of effect than the other three groups. In the second 7-day period, with the disturbance of some known external factors, such as the students' frequent outdoor activities and individual life styles, there were only 23% of the subjects still seeing the whitening effect. Regarding the two-week continuous use, there were still 41% of the subjects seeing the reduction of melanin.

By comparing the samples of Groups A, B, C and D, it is found that after the first week of use, the samples of Group D containing tranexamic acid, the tyrosinase inhibitor (CitrusC) and ascorbic acid 2-glucoside (AA2G), each in the amount of 0.1-2.5%, provided the best whitening effect, followed by Group C that contained only ascorbic acid 2-glucoside (AA2G). In the second 7-day period, the samples of Group A and Group C performed better. When it comes to the continuous use for two successive weeks, Group C provided the highest positive effect, i.e. 54%, followed by Group B, i.e. 46%, while the Group A brought up the rear. The data reveal that the samples of Group D containing tranexamic acid, the tyrosinase inhibitor (CitrusC) and ascorbic acid 2-glucoside (AA2G), each in the amount of 0.1-2.5%, provided the synergistic whitening effect. The composition presented the best whitening effect in the first week and remained effective in reducing melanin after two-week continuous use.

As to the commercially available products used in the experiment, namely Vital Purity Lotion (Group E) and CyberWhite Brilliant Cells Full Spectrum Brightening Essence (Group F), Group E gave unexpected results. After the first week of use, 38% of the subjects in Group E witnessed whitening effect, and in the second week of use, reduction in melanin exhibited in nearly 25% more of the subjects. Two-week continuous use made 63% of the subjects feel the whitening effect. Group F showed a positively affected population of 65% in the first week, and only 41% of the subjects felt the lasting whitening effect in the second week, yet the continuous use for the two whole weeks performed 10-30% better than the other groups. The figures indicate that the samples of Group F showed powerful whitening effect after the first week of use, with 65% of the subjects experienced the positive effect, and similar to that obtained after the first week of use of Group D.

TABLE 4
Whitening Effect of Differently Formulated Samples

| | Subject | | | |
|---|---|---|---|---|
| | Size | Positive Effect (%) | | |
| Group | (Person) | T0-7* | T7-14* | T0-14* |
| (A) Moisturizing Essence Base | 20 | 40 | 60 | 35 |
| + Tranexamic Acid | | | | |
| (B) Moisturizing Essence Base | 24 | 42 | 46 | 46 |
| + Tyrosinase Inhibitor | | | | |
| (Citrus C) | | | | |
| (C) Moisturizing Essence Base | 24 | 50 | 58 | 54 |
| + Ascorbic Acid 2-Glucoside | | | | |
| (AA2G) | | | | |
| (D) Moisturizing Essence Base | | | | |
| + Tranexamic Acid + Ascorbic | 39 | 62 | 23 | 41 |
| Acid 2-Glucoside (AA2G) + | | | | |
| Tyrosinase Inhibitor (Citrus C) | | | | |
| (E) Vital Purity Lotion (Kose) | 16 | 38 | 63 | 63 |
| (F) CyberWhite Brilliant Cells | 17 | 65 | 41 | 65 |
| Full Spectrum Brightening | | | | |
| Essence (Estée Laude) | | | | |

*T0-7 covering the seven days of the firstweek from the commencement of using the sample, T7-14 covering the seven days of the second week from the commencement of using the sample, T0-14 covering the fourteen days of the consecutive two weeks from the commencement of using the sample

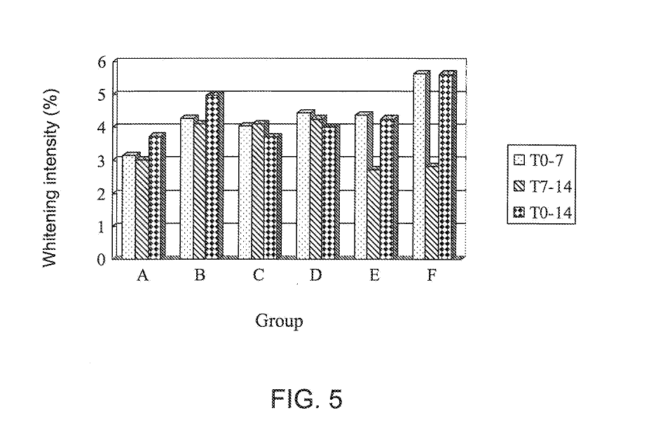

The trends of the whitening levels (indexes) of different groups can be seen clearly in Table 5 and FIG. 5. After the use of the samples of the groups for one week, the resultant whitening levels of the groups were consistent with the inventor's expatiation. Groups A and D showed comparable effects, while Group B was the least effective one despite its inclusion of the tyrosinase inhibitor (CitrusC). Group C containing ascorbic acid 2-glucoside (AA2G) showed some whitening effect but was not as effective as the samples containing tranexamic acid. The figures associated with Group D indicate that the combination of tranexamic acid, the tyrosinase inhibitor (CitrusC) and ascorbic acid 2-glucoside (AA2G) was more effective than any of these ingredients used alone. The synergy was particularly significant in the first week of use.

TABLE 5
Whitening Effect of Differently Formulated Samples
for Different Duration

| | Whitening intensity (%) | | |
|---|---|---|---|
| Group | T0-7* | T7-14* | T0-14* |
| (A) Moisturizing Essence Base + | 3.98 | 3.64 | 4.34 |
| Tranexamic Acid | | | |
| (B) Moisturizing Essence Base + | 2.85 | 5.29 | 5.20 |
| Tyrosinase Inhibitor (Citrus C) | | | |
| (C) Moisturizing Essence Base + | 3.39 | 3.76 | 3.15 |
| Ascorbic Acid 2-Glucoside | | | |
| (AA2G) | | | |
| (D) Moisturizing Essence Base + | 3.95 | 3.02 | 2.36 |
| Tranexamic Acid + Ascorbic | | | |
| Acid 2-Glucoside (AA2G) + | | | |
| Tyrosinase Inhibitor (Citrus C) | | | |
| (E) Vital Purity Lotion (Kose) | 3.54 | 4.59 | 3.73 |
| (F) CyberWhite Brilliant Cells | 4.79 | 5.32 | 5.46 |
| Full Spectrum Brightening | | | |
| Essence (Estée Laude) | | | |

* T0-7 covering the seven days of the first week from the commencement of using the sample, T7-14 covering the seven days of the second week from the commencement of using the sample, T0-14 covering the fourteen days of the consecutive two weeks from the commencement of using the sample

### IV. CONCLUSION

Group D in virtue of the joint addition of tranexamic acid, the tyrosinase inhibitor (CitrusC) and ascorbic acid 2-glucoside (AA2G), each in the amount of 0.1-2.5%, provided the whitening effect enhanced by the synergistic effect among the ingredients, and thus presented the best effect in the first week of use. Even after the use of two consecutive weeks, it still performed well in reducing melanin.

The composition of the present invention uses the tyrosinase inhibitor (CitrusC) to inhibit the action of tyrosinase directly, so as to prevent synthesis of melanin and facilitate skin whitening. Different from the conventional skin-whitening products, the disclosed composition has the tyrosinase inhibitor (CitrusC) acting as a catalyst for chemical reaction only, without acting with and being consumed by tyrosinase. Thus, the tyrosinase inhibitor (CitrusC) can provide sufficient and long-lasting whitening effect at a small dosage or low concentration.

Therein, ascorbic acid 2-glucoside is a derivative of Vitamin C and has been proven in lab as being effective in reducing synthesis of melanin. However, it is less absorbable to human skin because of its high molecular weight. As demonstrated in the conducted human subject research, the sole addition of ascorbic acid 2-glucoside did not show good whitening effect. However, since it is relatively easy to obtain, it is used in the present invention to protect isoflavone of the tyrosinase inhibitor (CitrusC) from oxidization, thereby facilitating the storage of the disclosed composition.

In addition, the tyrosinase inhibitor (CitrusC) is a natural extract, and the protein it contains may bring about allergic reaction when touching human skin. Therefore, the present invention also employs tranexamic acid, which has been greatly commercialized and highly available, for the purposes of preventing allergy and irritation. Conventionally, tranexamic acid can only give whitening effect with a concentration higher than 3%. As the present invention only uses it for preventing allergy and irritation but not whitening, the amount used here is only 0.1-2.5%.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. A skin-whitening essence composition containing a tyrosinase inhibitor, the skin-whitening essence composition primarily comprising: 0.1-2.5 wt % of the tyrosinase inhibitor (CitrusC); 0.1-2.5 wt % of ascorbic acid 2-glucoside (AA2G); 0.1-2.5 wt % of tranexamic acid; and a moisturizing essence base, that work synergistically on whitening skin.

2. The skin-whitening essence composition of claim 1, wherein the moisturizing essence base includes at least one moisturizing component.

3. The skin-whitening essence composition of claim 2, wherein the moisturizing component is selected from the group consisting of aloe vera extract, olive oil PEG-7 esters, γ-polyglutamic acid, lavender flower extract and combination thereof.

4. The skin-whitening essence composition of claim 1, wherein the tyrosinase inhibitor (CitrusC) comprises the following characteristics:
inhibiting tyrosinase effectively at 37° C.; and
functioning best under a pH value between 7.0 and 9.0.

5. The skin-whitening essence composition of claim 1, wherein the tyrosinase inhibitor (CitrusC) is obtainable by a process comprising steps of:
(1) mincing lemon peels;
(2) well mixing the minced lemon peels and propylene glycol in a ratio from 1:1 to 1:10 into a mixture, wherein the minced lemon peels are counted by weight (grams), and the propylene glycol is counted by volume (milliliters);
(3) breaking cell walls of the minced lemon peels in the mixture;
(4) sterilizing the mixture after the step (3); and
(5) performing centrifugation to the mixture and obtaining a supernate of the mixture as the tyrosinase inhibitor (CitrusC).
